(19) United States
(12) Patent Application Publication
(10) Pub. No.: US 2022/0323326 A1
(43) Pub. Date: Oct. 13, 2022

(54) KIT-OF-PARTS AND COMPOSITION FOR DYEING OF KERATIN FIBERS, METHOD, AND USE THEREOF

(71) Applicant: Kao Corporation, Tokyo (JP)

(72) Inventors: Bernd NÖCKER, Darmstadt (DE); Steven BREAKSPEAR, Darmstadt (DE); Fariba GHIASI, Darmstadt (DE); Anna NEU, Darmstadt (DE)

(73) Assignee: Kao Corporation, Tokyo (JP)

(21) Appl. No.: 17/634,317

(22) PCT Filed: Aug. 13, 2020
(86) PCT No.: PCT/EP2020/072747

§ 371 Date: Feb. 10, 2022

(30) Foreign Application Priority Data
Aug. 14, 2019 (EP) 19191691.5

(51) Int. Cl. A61K 8/41; A61K 8/22; A61K 8/43; A61K 8/46; A61K 8/23; A61Q 5/06

(52) U.S. Cl. CPC A61K 8/41; A61K 8/22; A61K 8/43; A61K 8/463; A61K 8/23; A61Q 5/065; A61K 2800/4322; A61K 2800/882; A61K 2800/48

(57) ABSTRACT

The present invention is directed to the provision of a kit-of-parts, a dyeing composition, and a method for dyeing keratin fibres. The kit comprises separate compositions A and B and optionally separate composition C. Composition A comprises a direct dye and an alkalising agent. The aqueous composition B comprises guanidine and/or its salts together with one or more lipophilic compounds and/or one or more thickening polymers. Composition C comprises a persalt and/or persulfate salt. A method for dyeing keratin fibres comprises mixing compositions A and B, and optionally C, to yield a ready-to-use composition with pH in the range of 7 to 12. Composition B may be used for enhancing colour intensity and/or wash fastness of a direct dye on keratin fibres with the provision that it is mixed with composition A directly prior to application.

### FIELD OF THE INVENTION

The present invention is directed to a kit-of-parts for dyeing of keratin fibers, as well as a method of dyeing and use of a composition. Furthermore, two-part and three-part dyeing compositions are disclosed.

### BACKGROUND OF THE INVENTION

A common approach to confer color to keratin fibers is the use of direct dyes. Such dyes have the advantage over oxidative dyes that the dyeing process leads to less damage of the keratin fibers. Moreover, the end-user as well as the professional hairdresser can estimate the resulting color of hair much better than with oxidative dyes before application of the dyeing composition to hair, because the color of the dyeing composition closely resembles the resulting hair color. As a result, the appearance is cosmetically more attractive and predictable, especially after repeated dyeing cycles.

A general problem with direct dyes is their unsatisfactory dyeing property on keratin fibers as well as their low wash fastness. The prior art has addressed these problems, but only partially delivered a satisfactory solution.

FR3060351, FR3060317, FR3060313, and JP2012126661 disclose oxidizing compositions for bleaching and dyeing of keratin fibers which comprise guanidine compounds. Guanidine compounds are disclosed to catalyze polymerization of oxidative dyes. However, the present invention is directed to direct dyes and has different technical effects as laid out below.

DE29722990, EP2883570, and EP2883571 disclose guanidine compounds in bleaching powder. However, the present invention is directed to an aqueous composition B.

### SUMMARY OF THE INVENTION

Thus, the first object of the present invention is a kit-of-parts for dyeing of keratin fibers, preferably human keratin fibers, more preferably human hair, comprising two or more separate compositions characterized in that
one separate composition is composition A comprising one or more direct dye(s) and one or more alkalizing agent(s),
one separate composition is composition B being an aqueous composition having a pH in the range of 1 to less than 6 comprising:
a) one or more guanidine compound(s) and/or its salts,
b) one or more lipophilic compound(s) and/or one or more thickening polymer(s), and
c) optionally one or more oxidizing agent(s).

The second object of the present invention is a three-part dyeing composition comprising composition A as defined above, composition B as defined above, and composition C comprising one or more persalt(s) and/or persulfate salt(s).

The third object of the present invention is a method for dyeing of keratin fibers, preferably human keratin fibers, more preferably human hair, comprising the steps of:
i) providing composition A as defined above,
ii) providing composition B as defined above,
iii) optionally providing composition C as defined above,
iv) mixing the compositions of i), ii), and optionally iii) directly prior to use to yield a ready-to-use composition having a pH in the range of 7 to 12,
v) applying the ready-to-use composition onto keratin fibers for a time period of 1 min to 60 min,
vi) optionally rinsing off the keratin fibers.

The fourth object of the present invention is a use of a composition as defined as composition B above for enhancing color intensity and/or wash fastness of one or more direct dye(s) on keratin fibers, preferably human keratin fibers, more preferably human hair with the provision that composition B is mixed with a composition A as defined above directly prior to application onto keratin fibers.

### DETAILED DESCRIPTION OF THE INVENTION

After long and considerate research, inventors of the present invention have found out that a kit-of-parts comprising a composition B having a pH in the range of 1 to less than 6 comprising one or more guanidine compound(s), one or more lipophilic compounds, and optionally an oxidizing agent provides for more intensive and durable color of direct dyes on keratin fibers being separated in a composition A. Furthermore, by using guanidine compound(s) in composition B, the storage stability of the direct dyes is enhanced, as direct dyes have the tendency to precipitate in presence of guanidine compound(s).

Without being bound by any theory, it is believed that guanidine and/or its salt(s) enhances penetration of direct dyes into hair fibers, and “lock” them into the hair fibers by weak molecular interactions. As a result, better dyeing efficiency and enhanced wash fastness was observed by the inventors of the present invention.

Composition A

Composition A comprises one or more direct dye(s) and one or more alkalizing agent(s).

Suitable direct dyes are selected from non-ionic, anionic, and/or cationic direct dyes.

Non-limiting examples of non-ionic direct dyes are those of neutral nitro dyes. Suitable non-limiting examples are HC Blue 2, HC Blue 4, HC Blue 5, HC Blue 6, HC Blue 7, HC Blue 8, HC Blue 9, HC Blue 10, HC Blue 11, HC Blue 12, HC Blue 13, HC Brown 1, HC Brown 2, HC Green 1, HC Orange 1, HC Orange 2, HC Orange 3, HC Orange 5, HC Red BN, HC Red 1, HC Red 3, HC Red 7, HC Red 8, HC Red 9, HC Red 10, HC Red 11, HC Red 13, HC Red 54, HC Red 14, HC Violet BS, HC Violet 1, HC Violet 2, HC Yellow 2, HC Yellow 4, HC Yellow 5, HC Yellow 6, HC Yellow 7, HC Yellow 8, HC Yellow 9, HC Yellow 10, HC Yellow 11, HC Yellow 12, HC Yellow 13, HC Yellow 14, HC Yellow 15,2-Amino-6-chloro-4-nitrophenol, picramic acid, 1,2-Diamino-4-nitrobenzol, 1,4-Diamino-2-nitrobenzol, 3-Nitro-4-aminophenol, 1-Hydroxy-2-amino-3-nitrobenzol and 2-hydroxyethylpicramic acid. Further non-limiting examples are direct dyes being neutral in aqueous solutions having a pH in the range of 7 to 12, for example HC Blue 18, HC Red 18, and HC Yellow 16.

Non-limiting examples of cationic dyes are Basic Blue 6, Basic Blue 7, Basic Blue 9, Basic Blue 26, Basic Blue 41, Basic Blue 99, Basic Brown 4, Basic Brown 16, Basic Brown 17, Basic Orange 31, Natural Brown 7, Basic Green 1, Basic Red 2, Basic Red 12 Basic Red 22, Basic Red 51, Basic Red 76, Basic Violet 1, Basic Violet 2, Basic Violet 3, Basic Violet 10, Basic Violet 14, Basic Yellow 57 and Basic Yellow 87.

Non-limiting examples of anionic direct dyes are Acid Black 1, Acid Blue 1, Acid Blue 3, Food Blue 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet 2, D&C Yellow No. 7, D&C Yellow No. 8, D&C Yellow No. 10, FD&C Red 2, FD&C Red 40, FD&C Red No. 4, FD&C Yellow No. 6, FD&C Blue 1, Food Black 1, Food Black 2, Disperse Black 9 and Disperse Violet 1 and their alkali metal salts such as sodium, potassium.

The preferred direct dyes of composition A are HC Blue 18, HC Red 18, and HC Yellow 16, and/or their mixtures, and/or their salt(s) from the viewpoint of color intensity and color brilliance.

It is preferred that the total concentration of direct dyes in composition A is 0.01% by weight or more, preferably 0.1% by weight or more, more preferably 0.25% by weight or more, calculated to the total weight of composition A, from the viewpoint of enhancing color intensity on keratin fibers.

It is preferred that the total concentration direct dyes in composition A is 10% by weight or less, preferably 8% by weight or less, more preferably 5% by weight or less, calculated to the total weight of composition A, from the viewpoint of enhancing color intensity on keratin fibers, dye solubility, and economic considerations.

For attaining the above-mentioned effects, it is preferred that the total concentration of direct dyes in composition A is in the range of 0.01% to 10% by weight, preferably in the range of 0.1% to 8% by weight, more preferably in the range of 0.25% to 5% by weight, calculated to the total weight of composition A.

Composition A comprises one or more alkalizing agent, suitably selected from ammonia and/or its salt(s), sodium metasilicate, 2-amino-2-methylpropanol, 2-amino-2-hydroxymethyl-propane-1,3-diol, organic alkyl and/or alkanol amine and/or their salt(s) according to the general structure

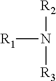

wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H.

The organic alkyl and/or alkanol amine according to the structure above may be selected from mono- and/or diethanolamine, butyl ethanolamine, butyl diethanolamine, dibutyl ethanolamine, methylethanolamine, triethanolamine, N-lauryl diethanolamine, diisopropanolamine, dimethyl isopropanolamine, isopropanolamine, triisopropanolamine, isobutanolamine, monoethylamine, diethylamine, trimethylamine.

The most preferred organic amine is mono- or diethanolamine from the viewpoint of formulation flexibility and absence of unpleasant smells.

The most preferred alkalizing agents from the viewpoint of dyeing intensity are ammonia and/or its salt(s) and 2-amino-2-methylpropanol.

The total concentration of alkalizing agents of composition A is 0.5% by weight or more, preferably 1% by weight or more, further more preferably 2% by weight or more, calculated to the total of composition A, from the viewpoint of achieving sufficient alkalinity in the ready-to-use mixture.

The total concentration of alkalizing agents of composition A is 30% by weight or less, preferably 20% by weight or less, further more preferably 15% by weight or less, from the viewpoint of achieving sufficient alkalinity in the ready-to-use mixture and economic reasons.

For attaining the above-mentioned effects, the total concentration of alkalizing agents in composition A is in the range of 0.5% to 30% by weight, preferably 1% to 20% by weight, more preferably 2% to 15% by weight, calculated to the total of composition A.

Composition A may be in any cosmetically suitable form, such as an aqueous composition, a liquid non-aqueous composition, or a powder composition.

In case composition A is an aqueous composition, the pH of the composition is in the range of 7 to 12, preferably in the range of 8 to 11, more preferably in the range of 8.5 to 10, from the viewpoint achieving an alkaline pH in the ready-to-use mixture.

In case composition A is a liquid non-aqueous composition, the composition is a solution of dyes in one or more appropriate organic solvents, as disclosed in WO2018087203.

In case composition A is a powder composition, the composition comprises additional powder ingredients as disclosed in WO2019057829.

It is preferred from the viewpoint of dye stability that composition A is free of persalt(s) and/or persulfate salt(s).

It is optional that composition A further comprises one or more thickening polymer(s), one or more lipophilic compound(s), or one or more surfactant(s), as also laid out below for composition B.

Composition B

Composition B is an aqueous composition having a pH in the range of 1 to less than 6 comprising:
a) one or more guanidine compound(s) and/or its salts,
b) one or more lipophilic compound(s) and/or one or more thickening polymer(s), and
c) optionally one or more oxidizing agent(s).

It is preferred from the viewpoint of cosmetic safety that the pH of the composition B is in the range of 2 to 5, more preferably in the range of 2.5 to 4.5.

It is preferred from the viewpoint of composition stability that composition B is free of persalt(s) and/or persulfate salt(s).

Although it is preferred that composition B does not comprise any direct dyes from the viewpoint of composition stability, the presence of one or more direct dyes is not excluded. However, the skilled person has to carefully select suitable direct dyes by their compatibility with the composition B. Suitable selection criteria are storage stability of composition B, and stability upon mixing to form the ready-to-use composition.

Compounds According to a)

The composition of the present invention comprises one or more guanidine compound(s) and/or its salts as compounds according to a).

Preferably, the compounds according to a) are selected from guanidine, guanidine carbonate, guanidine hydrochloride, guanidine sulfate, and guanidine phosphate, and/or their mixtures, from the viewpoint of enhancing color intensity on keratin fibers. The most preferred compound according to a) is guanidine sulfate, from the viewpoint of enhancing color intensity on keratin fibers as well as enhancing wash fastness of the direct dyes.

In one aspect of the present invention, guanidine sulfate may be formed in-situ by adding sulfuric acid to any other guanidine compound listed above, except for guanidine sulfate itself.

It is preferred that the total concentration of compounds according to a) in composition B is 0.1% by weight or more, preferably 0.5% by weight or more, more preferably 1% by weight or more, calculated to the total weight of the composition B, from the viewpoint of enhancing color intensity on keratin fibers.

It is preferred that the total concentration of compounds according to a) in composition B is 10% by weight or less, preferably 8% by weight or less, more preferably 6% by weight or less, calculated to the total weight of the composition B, from the viewpoint of enhancing color intensity on keratin fibers and compound solubility.

For attaining the above-mentioned effects, it is preferred that the total concentration of compounds according to a) in composition B is in the range of 0.1% to 10% by weight, preferably in the range of 0.5% to 8% by weight, more preferably in the range of 1% to 6% by weight, calculated to the total weight of the composition B.

Compounds According to b)

The composition of the present invention comprises one or more lipophilic compound(s) and/or one or more thickening polymer(s) as compounds according to b).

Suitably, the lipophilic compounds according to b) are selected from branched or linear, saturated or unsaturated C₁₂-C₂₂ fatty alcohols, esters of branched or linear C₃-C₁₂ alcohols with linear or branched, saturated or unsaturated C₈-C₂₂ fatty acids, and petrolatum-based products, and/or their mixtures, from the viewpoint of their non-reactivity with dyeing components and from the viewpoint of cosmetic compatibility.

Preferably, the petrolatum-based products are selected from mineral oil, paraffinum perliquidum, paraffinum subliquidum, and/or paraffinum solidum, and/or their mixtures, from the viewpoint of their non-reactivity with dyeing components.

Suitably, branched or linear, saturated or unsaturated C₁₂-C₂₂ fatty alcohols are lauryl alcohol, tridecyl alcohol, myristyl alcohol, pentadecyl alcohol, cetyl alcohol, palmitoleyl alcohol, heptadecyl alcohol, stearyl alcohol, oleyl alcohol, nonadecyl alcohol, arachidyl alcohol, behenyl alcohol, and/or their mixtures. Such a mixture is cetearyl alcohol comprising stearyl alcohol and cetyl alcohol.

The most preferred lipophilic compounds according to b) are cetearyl alcohol and/or mineral oil, from the viewpoint of cosmetic compatibility.

It is preferred that the total concentration of lipophilic compounds according to b) in composition B is 0.5% by weight or more, preferably 1% by weight or more, more preferably 2% by weight or more, calculated to the total weight of the composition B, from the viewpoint of enhancing color intensity on keratin fibers.

It is preferred that the total concentration of lipophilic compounds according to b) in composition B is 20% by weight or less, preferably 15% by weight or less, more preferably 12% by weight or less, calculated to the total weight of the composition B, from the viewpoint of enhancing color intensity on keratin fibers and formulating a stable cosmetic composition.

For attaining the above-mentioned effects, it is preferred that the total concentration of lipophilic compounds according to b) in composition B is in the range of 0.5% to 20% by weight, preferably in the range of 1% to 15% by weight, more preferably in the range of 2% to 12% by weight, calculated to the total weight of the composition B.

Suitably, thickening polymers according to b) are selected from polymers, which are able to produce a viscosity of 500 mPas in a 1% by weight aqueous solution within the desired pH range of 1 to less than 6.

Suitable polymers are, for example, xanthan gum, dehydroxanthan gum, methyl cellulose, ethyl cellulose, hydroxymethyl cellulose, and hydroxyethylcellulose, and/or their mixtures.

It is preferred from the viewpoint of viscosity and convenience that the total concentration of thickening polymers according to b) of composition B is 0.1% by weight or more, more preferably 0.2% by weight or more, further more preferably 0.5% by weight or more, calculated to the total weight of the composition B.

It is preferred from the viewpoint of viscosity and convenience that the total concentration of thickening polymers according to b) of composition B is 10% by weight or less, more preferably 8% by weight or less, further more preferably 5% by weight or less, calculated to the total weight of the composition B.

For attaining the above-mentioned effects, it is preferred that the total concentration of thickening polymers according to b) of composition B is in the range of 0.1% to 10% by weight, preferably in the range of 0.2% to 8% by weight, more preferably in the range of 0.5% to 5% by weight, calculated to the total weight of the composition B.

It is to be noted that the skilled person may combine lipophilic compounds and thickening polymers according to b). With both measures, it is possible to thicken the composition B to achieve the desired technical effect. In this case, the skilled person will select appropriate ratios of lipophilic compounds and thickening polymers.

Compounds According to c)

The composition of the present invention optionally comprises one or more oxidizing agent(s).

Preferably, the compound according to c) is hydrogen peroxide, from the viewpoint of achieving lightening of the keratin fibers during the dyeing process.

It is preferred that the total concentration of compounds according to c) in composition B, preferably hydrogen peroxide, is 0.5% by weight or more, preferably 1% by weight or more, more preferably 2% by weight or more, calculated to the total weight of the composition B, form the viewpoint of achieving sufficient lightening.

It is preferred that the total concentration of compounds according to c) in composition B, preferably hydrogen peroxide, is 20% by weight or less, preferably 15% by weight or less, more preferably 12% by weight or less, calculated to the total weight of the composition B, form the viewpoint of limiting damage on keratin fibers.

For attaining the above-mentioned effects, the total concentration of compounds according to c) in composition B, preferably hydrogen peroxide, is in the range of 0.5% to 20% by weight, preferably in the range of 1% to 15% by weight, more preferably in the range of 2% to 12% by weight, calculated to the total weight of the composition B.

However, if lightening of the keratin fibers is less desired, the composition of the present invention does not comprise an oxidizing agent, preferably hydrogen peroxide.

Compounds According to d)

Composition B may comprise one or more surfactant(s) as compounds according to d), preferably selected from anionic, non-ionic, cationic and/or amphoteric/zwitterionic surfactants, and/or their mixtures, from the viewpoint of enhancing the stability of the composition.

Preferably, the total concentration of compounds according to d) in composition B is 0.05% by weight or more, more preferably 0.1% by weight or more, further more preferably 0.2% by weight or more, calculated to the total weight of composition B, from the viewpoint of improving emulsion stability.

Preferably, the total concentration of compounds according to d) in composition B is 15% by weight or less, more preferably 10% by weight or less, still more preferably 5% by weight or less, calculated to the total weight of composition B, from the viewpoint of emulsion stability and cost of goods aspect.

For attaining the above-mentioned effect, preferably the total concentration of compounds according to d) in composition B is in the range of 0.05% to 15% by weight, preferably 0.1% to 10% by weight, more preferably 0.2% to 5% by weight, calculated to the total weight of composition B.

Suitable compounds according to d) are disclosed below.

The preferred compound d) are anionic surfactants and/or non-ionic surfactants from the viewpoint of emulsion stabilization properties.

Anionic Surfactants

Preferably, the anionic surfactants are selected from ethoxylated or non-ethoxylated alkyl ether sulfate surfactants, alkyl sulfates, ethoxylated and/or non-ethoxylated alkyl carboxylates, ethoxylated or non-ethoxylated amino acid surfactants, and/or their mixtures, from the viewpoint of their emulsion stabilization properties.

Suitable alkyl sulfate or preferably ethoxylated alkyl ether sulfate surfactant or mixtures thereof have an alkyl chain length of C₁₀ to C₂₂.

Suitable example alkyl sulfate surfactants are lauryl sulfate, myristyl sulfate, olel sulfate, and behenyl sulfate.

Suitable example alkyl ether sulfate surfactants are laureth sulfates, coceth sulfate, pareth sulfate, capryleth sulphate, myreth sulfate, oleth sulfate, deceth sulfate, trideceth sulfate, coco sulphate, C₁₀-C₁₆ alkyl sulphate, C₁₁-C₁₅ alkyl sulphate, C₁₂-C₁₈ alkyl sulphate, C₁₂-C₁₅ alkyl sulphate, C₁₂-C₁₆ alkyl sulphate, C₁₂-C₁₃ alkyl sulfate, lauryl sulphate, myristyl sulphate, palm kernel sulphate, cetearyl sulfate, cetyl sulphate, decyl sulphate, oleyl sulphate, behenyl sulphate and/or their salts. All of the aforementioned anionic surfactants may or may not be ethoxylated at various degrees.

Cations for all of the above-mentioned surfactants may be selected from sodium, potassium, magnesium and/or ammonium.

The most preferred anionic surfactant is sodium lauryl sulfate, from the viewpoint of emulsions stabilization.

Non-Ionic Surfactants

Suitable non-ionic surfactants are selected from alkyl polyglycosides, ethoxylated triglycerides, ethoxylated fatty alcohols, and/or their mixtures.

Suitable examples of alkyl polyglycosides are decyl glucoside, lauryl glucoside, and coco glucoside.

Further suitable nonionic surfactants are ethoxylated triglycerides. Well-known and commonly used examples are ethoxylated castor oil such as PEG-40 hydrogenated castor oil or and PEG-60 hydrogenated castor oil.

Suitable examples of ethoxylated fatty alcohols are C9-11 Pareth-6, C9-11 Pareth-8, C9-15 Pareth-8, C11-13 Pareth-9, C11-13 Pareth-10, C11-15 Pareth-5, C11-15 Pareth-7, C11-15 Pareth-9, C11-15 Pareth-12, C11-15 Pareth-15, C11-15 Pareth-20, C11-15 Pareth-30, C11-15 Pareth-40, C11-21 Pareth-10, C12-13 Pareth-5, C12-13 Pareth-6, C12-13 Pareth-7, C12-13 Pareth-9, C12-13 Pareth-10, C12-13 Pareth-15, C12-13 Pareth-23, C12-14 Pareth-5, C12-14 Pareth-7, C12-14 Pareth-9, C12-14 Pareth-11, C12-14 Pareth-12, C12-15 Pareth-5, C12-15 Pareth-7, C12-15 Pareth-9, C12-15 Pareth-10, C12-15 Pareth-11, C12-15 Pareth-12, C12-16 Pareth-5, C12-16 Pareth-7, C12-16 Pareth-9, C13-15 Pareth-21, C14-15 Pareth-7, C14-15 Pareth-8, C14-15 Pareth-11, C14-15 Pareth-12, C14-15 Pareth-13, C20-22 Pareth-30, C20-40 Pareth-10, C20-40 Pareth-24, C20-40 Pareth-40, C20-40 Pareth-95, C22-24 Pareth-33, Beheneth-5, Beheneth-10, Beheneth-15, Beheneth-20, Beheneth-25, Beheneth-30, Ceteareth-5, Ceteareth-6, Ceteareth-7, Ceteareth-10, Ceteareth-11, Ceteareth-12, Ceteareth-15, Ceteareth-20, Ceteareth-25, Ceteareth-30, Ceteareth-35, Ceteareth-40, Laureth-5, Laureth-10, Laureth-15, Laureth-20, Laureth-25, Laureth-30, Laureth-40, Myreth-5, Myreth-10, Ceteth-5, Ceteth-10, Ceteth-15, Ceteth-20, Ceteth-25, Ceteth-30, Ceteth-40, Oleth-5, Oleth-10, Oleth-15, Oleth-20, Oleth-25, Oleth-30, Oleth-40, Steareth-5, Steareth-10, Steareth-15, Steareth-20, Steareth-25, Steareth-30, Steareth-35, and Steareth-40.

Amphoteric/Zwitterionic Surfactants

Suitable compounds are known as hydroxysultaine surfactants, such as cocoamidopropyl hydroxysultaine, laurylamidopropyl hydroxysultaine, erucamidopropyl hydroxysultaine, lauryl hydroxysultaine, and cocoyl hydroxysultaine, and/or their salt(s).

Further suitable amphoteric/zwitterionic surfactants are of betaine type. Suitable compounds may be selected from alkyl betaines and/or alkylamido betaines. A preferred compound selected from alkyl betaines is lauryl betaine. A preferred compound selected from alkylamido betaines is cocamidopropyl betaine. The disclosure also relates to the salts of the compounds.

Cationic Surfactants

Suitable examples of cationic surfactants are cetyl trimethyl ammonium chloride, stearyl trimonium chloride, dipalmitoyl diimonium chloride, distearyl dimethyl ammonium chloride, stearamidopropyl trimethyl ammonium chloride, dioleoylethyl dimethyl ammonium methosulfate, dioleoylethyl hydroxyethylmonium methosulfate, behenyl trimethyl ammonium chloride.

Composition C

In one aspect of the present invention, the kit-of-parts comprises a composition C.

In such a case, it is preferred from the viewpoint of lightening hair color that composition C comprises one or more persalt(s) and/or persulfate salt(s).

From the viewpoint of composition stability, it is also preferred that in the latter case the composition C is a powder composition, preferably an anhydrous powder composition.

Suitable persalt(s) and/or persulfate salt(s) are sodium persulfate, potassium persulfate, and ammonium persulfate, earth alkali peroxides such as magnesium peroxide, melamine peroxide or urea peroxide or phtholimidoperoxy hexanoic acid. The preferred persalts from the viewpoint of commercial reasons and bleaching efficiency are sodium persulfate and potassium persulfate.

The total concentration of persalt(s) and/or persulfate salt(s) in composition C is 10% by weight or more, preferably 15% by weight or more, more preferably 20% by weight or more, calculated to the total weight of the composition, from the viewpoint of bleaching efficiency.

The total concentration of persalt(s) and/or persulfate salt(s) in composition C is 80% by weight or less, preferably 70% by weight or less, more preferably 60% by weight or less, calculated to the total weight of the composition, from the viewpoint of bleaching efficiency and powder dispersability.

For attaining the above-mentioned effects, the total concentration of persalt(s) and/or persulfate salt(s) in composition C is in the range of 10% to 80%, preferably 15% to 70%, more preferably 20% to 60% and most preferably 25% to 60% by weight, calculated to the total weight of composition C.

Composition C preferably further comprises one or more alkalizing agent(s) from the viewpoint of achieving sufficient alkalinity for bleaching in the ready-to-use composition. The preferred alkalizing agent is sodium metasilicate from the viewpoint of hygroscopicity. Suitable concentrations are in the range from 1% to 20% by weight, calculated to the total weight of composition C.

It is further to be noted that for achieving a sufficient bleaching effect on keratin fibers, the presence of hydrogen peroxide or other suitable oxidizing agents for bleaching is required. Thus, in that case, either composition B comprises hydrogen peroxide as of feature c), or the kit-of-parts of the present invention comprises an additional composition comprising hydrogen peroxide being kept separately stored until directly prior to use. Suitable concentrations of hydrogen peroxide are as laid out for feature c).

Mixing of Kit Components

It is preferred from the viewpoint of stability that the components of the kit are kept separate until directly prior to use.

Thus, it is preferred that the components of the kit are mixed directly prior to application onto keratin fibers.

Three-Part Composition

The present invention may be in the form of a three-part dyeing composition comprising a composition A as defined above, a composition B as defined above, and a composition C as defined above.

This aspect is particularly suitable for customers with dark hair who desire a high degree of lightening in combination with dyeing. In that case, feature c) of composition B must be present as well.

The three-part dyeing composition is kept separate until directly prior to use. After mixing the components in appropriate ratios, suitably from 1:1:0.1 by weight (composition A:B:C) to 1:2:0.5 by weight (composition A:B:C), a ready-to-use mixture is formed.

Method of Dyeing

The present invention is also directed to a method for dyeing of keratin fibers, preferably human keratin fibers, more preferably human hair, comprising the steps of:
i) providing a composition A as defined above,
ii) providing a composition B as defined above,
iii) optionally providing a composition C as defined above,
iv) mixing the compositions of i), ii), and optionally iii) directly prior to use to yield a ready-to-use composition having a pH in the range of 7 to 12,
v) applying the ready-to-use composition onto keratin fibers for a time period of 1 min to 60 min,
vi) optionally rinsing off the keratin fibers.

It is preferred for the method above that the direct dye(s) of step a) are selected from HC Blue 18, HC Red 18, and HC Yellow 16, from the viewpoint of color intensity and brightness.

It is also preferred that the pH of the ready-to-use composition of step iv) is in the range of 8 to 11, more preferably in the range of 8.5 to 10, from the viewpoint of conferring color intensity and minimizing hair damage.

It is further preferred that the ready-to-use composition in step v) is applied for 5 to 45 min, more preferably from 10 to 30 min, from the viewpoint of conferring color intensity and minimizing hair damage.

It is also preferred to rinse-off the ready-to-use composition in step vi) with water, suitably followed by a shampooing step, from the viewpoint of cosmetic safety.

Use of Composition

Another object of the present invention is a use of a composition as defined as composition B for dyeing of keratin fibers, preferably human keratin fibers, more preferably human hair, to enhance color intensity and/or wash fastness of one or more direct dye(s) with the provision that composition B is mixed with a composition A as defined above directly prior to application onto hair.

Composition B as defined above leads to a higher color intensity on keratin fibers which can be observed when comparing streaks of fibers having been dyed with the inventive composition vs. non-inventive compositions.

Furthermore, wash fastness of direct dyes is also improved.

The following examples are to illustrate the present invention, but not to limit it.

### EXAMPLES

### Examples 1-3

The following first composition was prepared by dissolving the dyestuff in a water comprising the organic amine. The final pH was adjusted after complete dissolution of all components.

| | % by weight |
|---|---|
| HC Blue 18 | 0.1 |
| HC Red 18 | 0.08 |
| HC Yellow 16 | 0.05 |
| Monoethanolamine | 2.0 |
| NaOH/HCl | q.s. ad pH 9 |
| Water | ad 100.0 |

The following second compositions were prepared by dissolving surfactant in water and, if present, adding the guanidine salts. The solution was then heated to 60° C. and mineral oil was added while stirring. After cooling, hydrogen peroxide solution was added and the final pH was adjusted with phosphoric acid.

| | | Inventive | Inventive | Comparative |
|---|---|---|---|---|
| | | ex. 1 [% | ex. 2 [% | ex. 3 [% |
| | Ingredient | by weight] | by weight] | by weight] |
| Composition | Guanidine sulfate | 3.0 | — | — |
| | Guanidine | — | 3.0 | — |
| | phosphate | | | |
| | Mineral oil | 2.0 | 2.0 | 2.0 |
| | Sodium lauryl | 0.2 | 0.2 | 0.2 |
| | sulfate | | | |
| | Phosphoric acid | q.s. ad pH 3.5 | | |
| | Water | Ad 100.0 | | |
| Effect | L* | 16.74 | 17.58 | 19.95 |
| | a* | 2.42 | 3.44 | 3.11 |
| | b* | 2.57 | −2.71 | 3.78 |
| | ΔE*_{ab} | 67.00 | 67.31 | 63.66 |

The untreated hair streak had the following values: L*82.76, a*1.02, and b*13.91.

The first composition and each of the second compositions were mixed in a weight ratio of 1:1 yielding a ready-to-use mixture having a pH of 9.

Inventive examples 1 and 2 illustrate all objects of the present invention.

Compositions A of working examples 4 to 7 and comparative examples 8 and 9 were prepared by dissolving 2-amino-methyl propanol in 1,2-propandiol first, then by adding the dyestuff. The mixture was stirred until complete dissolution. Compositions B were prepared as laid out for example 1-3.

| | | | Working examples | | | | Comparative examples | |
|---|---|---|---|---|---|---|---|---|
| | Ingredient | | 4 | 5 | 6 | 7 | 8 | 9 |
| Comp. A | HC Blue 18 | | 2.0 | 2.0 | — | — | 2.0 | — |
| | HC Yellow 16 | | — | — | 2.0 | 2.0 | — | 2.0 |
| | 2-Amino-methyl propanol | | 5.0 | 5.0 | 5.0 | 5.0 | 5.0 | 5.0 |
| | 1,2-propandiol | | Ad 100.0 | | | | | |
| Mix | Mixing ratio A:B by weight | | 1:10 | 1:10 | 1:10 | 1:10 | 1:10 | 1:10 |
| Comp. B | Guanidine sulfate | | 3.0 | — | 3.0 | — | — | — |
| | Guanidine phosphate | | — | 3.0 | — | 3.0 | — | — |
| | Mineral oil | | 4.0 | 4.0 | 4.0 | 4.0 | 4.0 | 4.0 |
| | Sodium lauryl sulfate | | 0.5 | 0.5 | 0.5 | 0.5 | 0.5 | 0.5 |
| | Hydrogen peroxide | | 6.0 | 6.0 | 6.0 | 6.0 | 6.0 | 6.0 |
| | Phosphoric acid | | q.s. ad pH 3.0 | | | | | |
| | Water | | Ad 100.0 | | | | | |
| Effect | Directly | L* | 24.83 | 23.64 | 73.23 | 71.75 | 27.69 | 75.25 |
| | upon | a* | 10.85 | 10.16 | 21.35 | 24.15 | 9.23 | 16.17 |
| | dyeing | b* | −29.74 | −26.92 | 83.04 | 84.63 | −26.57 | 77.66 |
| | | Δ E*_{ab} | 73.02 | 72.23 | 72.90 | 75.46 | 68.64 | 66.11 |
| | Upon 20x | L* | 30.84 | 33.26 | 76.81 | 77.94 | 34.34 | 79.22 |
| | washing | a* | 6.78 | 4.91 | 15.86 | 16.17 | 4.07 | 10.61 |
| | | b* | −24.20 | −19.66 | 75.11 | 77.66 | −16.40 | 67.24 |
| | | Δ E*_{ab} | 64.42 | 59.65 | 63.43 | 56.22 | 56.90 | 54.42 |
| | Fastness | ΔΔ E*_{ab} | 8.60 | 12.58 | 9.47 | 19.24 | 11.74 | 11.69 |

The ready-to-use mixture had a pH of 9.0 ± 0.5. The untreated hair streaks had the following values: L* 82.24, a* −0.24, and b* 14.00.

### Examples 10-13

The following compositions were prepared in analogy to the previous examples with the exception if comparative example 13. The latter was prepared by dissolving surfactant in water and adding the guanidine salts and 2-aminomethyl propanol. The solution was then heated to 60° C. and mineral oil was added while stirring. After cooling, phosphoric acid solution was added and the final pH was adjusted.

| | | Inventive | Inventive | Inventive | Comparative |
|---|---|---|---|---|---|
| | | ex. 10 | ex. 11 | ex. 12 | ex. 13 |
| | | [% by | [% by | [% by | [% by |
| | Ingredient | weight] | weight] | weight] | weight] |
| Composition B | 2-aminomethyl | — | — | — | 5.0 |
| | propanol | | | | |
| | HC Red 18 | — | — | 0.2 | 0.2 |
| | Guanidine sulfate | 3.0 | — | — | 3.0 |
| | Guanidine phosphate | — | 3.0 | 3.0 | — |
| | Mineral oil | 2.0 | 2.0 | 2.0 | 2.0 |
| | Sodium lauryl sulfate | 0.2 | 0.2 | 0.2 | 0.2 |
| | Phosphoric acid | q.s. ad pH 3.5 | | q.s. ad pH 3.5 | q.s. ad pH 9.5 |
| | Water | Ad 100.0 | | Ad 100.0 | Ad 100.0 |
| Effect | Stability after storage | Stable | Stable | Stable red | Red |
| | for 2 weeks at 40° C. | emulsion | emulsion | emulsion | precipitates |

### Examples 13-22

| | | | Inventive examples | | | | | | | | | |
|---|---|---|---|---|---|---|---|---|---|---|---|---|
| | Ingredient | | 14 | 15 | 16 | 17 | 18 | 19 | 20 | 21 | 22 | 23 |
| Comp. A | HC Blue 18 | | 0.2 | — | — | — | — | — | — | — | — | — |
| | HC Yellow 16 | | — | 0.2 | 0.2 | 0.2 | 0.2 | 0.2 | 0.2 | 0.2 | 0.2 | 0.2 |
| | Ammonia | | 3.85 | 3.85 | 3.85 | 3.85 | 3.85 | 3.85 | 3.85 | 3.85 | 3.85 | 3.85 |
| | Water | | Ad 100.0 | | | | | | | | | |
| Mix | Mixing ratio A:B by | | 1:1 | 1:1 | 1:1 | 1:1 | 1:1 | 1:1 | 1:1 | 1:1 | 1:1 | 1:1 |
| | weight | | | | | | | | | | | |
| Comp. B | Guanidine sulfate | | 0.5 | 0.5 | 3.0 | 3.0 | 3.0 | 3.0 | 3.0 | — | 3.0 | 0.5 |
| | Guanidine carbonate** | | — | — | — | — | — | — | — | 3.0 | — | — |
| | Sulfuric acid** | | — | — | — | — | — | — | — | 1.73 | — | — |
| | Mineral oil | | 4.0 | 4.0 | 4.0 | 4.0 | 1.0 | 15.0 | 4.0 | 4.0 | 4.0 | — |
| | Xanthan gum | | — | — | — | — | — | — | — | — | — | 1.0 |
| | Sodium lauryl sulfate | | 0.5 | 0.5 | 0.5 | 0.5 | 0.2 | 3.0 | 0.5 | 0.5 | 0.5 | 0.5 |
| | Lanette N* | | — | — | — | — | — | — | 2.0 | — | — | — |
| | Cetearyl alcohol | | 2.0 | 2.0 | 2.0 | 2.0 | 2.0 | 2.0 | — | 2.0 | 3.0 | 2.0 |
| | Hydrogen peroxide | | 6.0 | 6.0 | 6.0 | 6.0 | 6.0 | 6.0 | 6.0 | 6.0 | 6.0 | 6.0 |
| | Phosphoric acid | | q.s. ad pH 3.0 | | | | | | | | | |
| | Water | | Ad 100.0 | | | | | | | | | |
| Effect | Directly | L* | 27.63 | 73.41 | 72.42 | 73.73 | 71.31 | 71.51 | 72.39 | 72.17 | 72.01 | 73.10 |
| | upon | a* | 13.68 | 23.88 | 23.57 | 23.45 | 27.47 | 24.57 | 25.77 | 24.92 | 26.53 | 25.35 |
| | dyeing | b* | −34.48 | 88.14 | 87.64 | 86.97 | 87.22 | 82.99 | 86.55 | 84.17 | 84.76 | 84.77 |
| | | Δ E*_{ab} | 73.18 | 79.03 | 78.57 | 77.76 | 79.56 | 74.62 | 78.24 | 75.75 | 76.86 | 76.35 |

*Lanette N is a mixture of cetearyl alcohol with sodium cetylstearylsulfate
**Sulfate salt is formed in-situ

Methods

Hair Dyeing Experiments

Caucasian blond hair streaks were obtained from volunteers and merged to yield 2 g of hair per bundle. The streaks were the shampooed with a commercially available shampoo under the brand name Goldwell Dualsenses Scalp Specialist Deep Cleansing Shampoo, thoroughly rinsed, and allowed to air-dry completely. After complete air-drying of the hair streaks, the hair color was measured by spectrophotometrical analysis with a Datacolor 45G CT instrument obtained from Datacolor Inc., Lawrenceville, N.J., U.S.A. 5 measurements points on the hair streaks were averaged (L₁, a₁, b₁).

Then 1 g of the example compositions from above were applied onto the hair streaks, massaged for 1 min, and allowed to rest for 15 min. The streaks were then rinsed with lukewarm water for 1 min and blow-dried. The hair streaks were then combed through and color measurements of wet streaks were conducted at 5 different positions on the streak (L₂, a₂, b₂)

Based on the CIE*Lab color space results obtained by the measurements, Lab values as well as ΔE*_{ab} values for color difference were calculated according to the following equation:

ΔE_{a,b}=\sqrt{(L₂−L₁)²+(a₂−a₁)²+(b₂−b₁)²}

Wash Fastness

For investigating wash fastness, the dyed hair streaks were individually placed in a shaking bath comprising 10% by weight of a sodium laureth sulfate solution for 30 min at 40° C. and 100 rpm. This treatment simulates 20 shampooing cycles. After completion of the shaking bath treatment, the hair streaks were rinsed with lukewarm water for 1 min and blow-dried. The dried hair streaks were then measured on their color content as described above and ΔE*_{ab} was calculated. Additionally ΔΔE*_{ab} was calculated as being the difference between ΔE*_{ab} directly upon coloring and ΔE*_{ab} after the wash fastness experiment.

Stability Testing

Stability of the compositions were tested in a storage test under defined conditions at 40° C. for 2 weeks. The compositions were visually evaluated by the human operator.

pH Measurements

All pH measurements for the composition of the present invention were made with a calibrated glass electrode at 25° C. under atmospheric pressure.

The following examples are within the scope of the present invention.

### Inventive Example 24

Composition A

| | % by weight |
|---|---|
| HC Blue 18 | 0.1 |
| HC Red 18 | 0.05 |
| HC Yellow 16 | 0.08 |
| Monoethanolamine | 2.0 |
| Benzyl alcohol | 3.0 |
| Xanthan gum | 0.30 |
| NaOH/HCl | q.s. ad pH 9.0 |
| Water | ad 100.0 |

Composition B

| | % by weight |
|---|---|
| Guanidine sulfate | 3.0 |
| Mineral oil | 1.0 |
| Cetearyl alcohol | 1.0 |
| Sodium lauryl sulfate | 0.5 |
| Phosphoric acid | q.s. ad pH 3.0 |
| Hydrogen peroxide | 6.0 |
| Water | ad 100.0 |

Compositions A and B are mixed in a weight ratio 1:1 to yield a pH in the range of 8 to 9.

### Inventive Example 25

Composition A

| | % by weight |
|---|---|
| HC Blue 18 | 0.1 |
| HC Red 18 | 0.05 |
| HC Yellow 16 | 0.08 |
| Monoethanolamine | 2.0 |
| Benzyl alcohol | 3.0 |
| Xanthan gum | 0.30 |
| NaOH/HCl | q.s. ad pH 9.0 |
| Water | ad 100.0 |

Composition B

| | % by weight |
|---|---|
| Guanidine sulfate | 3.0 |
| Mineral oil | 1.0 |
| Cetearyl alcohol | 1.0 |
| Sodium lauryl sulfate | 0.5 |
| Phosphoric acid | q.s. ad pH 3.0 |
| Hydrogen peroxide | 6.0 |
| Water | ad 100.0 |

Composition C

| | % by weight |
|---|---|
| Potassium persulfate | 25.0 |
| Sodium persulfate | 25.0 |
| Sodium metasilicate | 15.0 |
| Diatomaceous earth | ad 100.0 |

Compositions A, B, and C are mixed in a weight ratio 1:1:0.2 to yield a pH in the range of around 9 to 9.5.

1: A kit-of-parts for dyeing of keratin fibers comprising two or more separate compositions, wherein
one separate composition is composition A comprising one or more direct dye(s) and one or more alkalizing agent(s), and
one separate composition is composition B being an aqueous composition having a pH in a range of 1 to less than 6, wherein the composition B comprises:
a) one or more guanidine compound(s) and/or its salts,
b) one or more lipophilic compound(s) and/or one or more thickening polymer(s), and
c) optionally one or more oxidizing agent(s).

2: The kit-of-parts according to claim 1, wherein the compositions A and/or B are free of persalt(s) and/or persulfate salt(s).

3: The kit-of-parts according to claim 1, wherein at least one of the direct dyes of composition A is at least one selected from the group consisting of HC Blue 18, HC Red 18, and HC Yellow 16.

4: The kit-of-parts according to claim 1, wherein the composition A is an aqueous composition, a liquid non-aqueous composition, or a powder composition.

5: The kit-of-parts according to claim 1, wherein the total concentration of direct dyes of composition A is in the range of 0.01% to 10% by weight, calculated to the total weight of composition A.

6: The kit-of-parts according to claim 1, wherein compounds according to a) of composition B are at least one selected from the group consisting of guanidine, guanidine carbonate, guanidine hydrochloride, guanidine sulfate, and guanidine phosphate.

7-17. (canceled)

18: The kit-of-parts according to claim 1, wherein the compound according to a) of composition B is guanidine sulfate.

19: The kit-of-parts according to claim 1, wherein the total concentration of compounds according to a) of composition B is in the range of 0.1% to 10% by weight, calculated to the total weight of the composition B.

20: The kit-of-parts according to claim 1, wherein the total concentration of lipophilic compounds according to b) of composition B is in the range of 0.5% to 20% by weight, calculated to the total weight of the composition B.

21: The kit-of-parts according to claim 1, wherein the total concentration of thickening polymers according to b) of composition B is in the range of 0.1% to 10% by weight, calculated to the total weight of the composition B.

22: The kit-of-parts according to claim 1, wherein the composition B comprises compounds according to c).

23: The kit-of-parts according to claim 1, wherein the composition B comprises hydrogen peroxide as the compound according to c).

24: The kit-of-parts according to claim 1, wherein the composition B further comprises one or more surfactant(s) as compounds according to d).

25: The kit-of-parts according to claim 1, wherein the pH of the composition B is in the range of 2 to 5.

26: The kit-of-parts according to claim 1, wherein the pH of the composition B is in the range of 2.5 to 4.5.

27: The kit-of-parts according to claim 1, wherein the components of the kit are mixed directly prior to application onto keratin fibers.

28: The kit-of-parts according to claim 1, which further comprises a composition C comprising one or more persalt(s) and/or persulfate salt(s).

29: A three-part dyeing composition, comprising compositions A and B according to claim 1, and composition C comprising one or more persalt(s) and/or persulfate salt(s).

30: A method for dyeing of keratin fibers, the method comprising:
i) providing a composition A,
ii) providing a composition B,
wherein the compositions A and B are defined in claim 1,
iii) optionally providing composition C comprising one or more persalt(s) and/or persulfate salt(s),
iv) mixing the compositions of i), ii), and optionally iii) directly prior to use to yield a ready-to-use composition having a pH in the range of 7 to 12,
v) applying the ready-to-use composition onto keratin fibers for a time period of 1 min to 60 min, and
vi) optionally rinsing off the keratin fibers.

31: A method for enhancing color intensity and/or wash fastness of one or more direct dye(s) on keratin fibers, the method comprising application of compositions A and B according to claim 1, wherein the composition B is mixed with the composition A directly prior to application onto keratin fibers.
